(19) United States
(12) Patent Application Publication
(10) Pub. No.: US 2005/0236579 A1
(43) Pub. Date: Oct. 27, 2005

(54) Methods for rapidly sterilizing a small object

(75) Inventors: Geoffrey H. Jenkins, Wellesley Hills, MA (US); Richard Eckhardt, Arlington, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: UV-Solutions, LLC, Wellesley Hills, MA (US)

(21) Appl. No.: 11/165,105

(22) Filed: Jun. 23, 2005

Related U.S. Application Data
(62) Division of application No. 10/017,475, filed on Dec. 14, 2001.
(60) Provisional application No. 60/255,555, filed on Dec. 14, 2000.

(51) Int. Cl. G01N 21/51; G01N 21/01; G01N 21/00

(52) U.S. Cl. 250/455.11; 250/453.11

(57) ABSTRACT

Methods of sterilizing or disinfecting an object are disclosed. According to one embodiment of the invention, a method comprises introducing at least a first portion of an object into a sterilizer/disinfector, sealing light within the sterilizer/disinfector using at least a second portion of the object to form a light seal, and automatically, upon detection of completion of the light seal to a certain degree, flashing ultraviolet light onto the at least first portion of the object within the sterilizer/disinfector.

### PRIORITY CLAIM

This application is a divisional of U.S. patent application Ser. No. 10/017,475, entitled “METHOD AND APPARATUS FOR RAPIDLY STERILIZING SMALL OBJECTS, filed Dec. 14, 2001, now pending, which claims the benefit, under 35 U.S.C. §119(e), of the filing date of U.S. provisional application Ser. No. 60/255,555 entitled “Method and Apparatus for Rapidly Sterilizing Small Objects,” filed Dec. 14, 2000, which is incorporated herein by reference.

### FIELD OF THE INVENTION

The present invention relates generally to the field of sterilization or disinfection systems and methods.

### BACKGROUND OF THE INVENTION

A number of small objects used in everyday life, particularly those used in medical and hygienic applications, can serve as a transport mechanism for disease-causing microorganisms. Objects that are handled or breathed-on by different people, or come in contact with surfaces contaminated by other people or animals, can themselves become contaminated. If these objects then contact another person, they can transmit diseases. Even the hands and clothing of medical or healthcare personnel can serve to transmit diseases.

This contamination problem is particularly acute with objects used in medical facilities or for hygienic applications, or the hands and clothing of workers in these facilities, as they have a much higher probability of contacting infected people or surfaces. Some medical devices are designed to be placed in contact with diseased patients. If they are not sterilized between use on different patients, they can serve as the vector to transmit the disease from one person to the next. Examples of this are thermometers, otoscopes, blood pressure meters, stethoscopes and other devices used by used by doctors, nurses, and other medical or healthcare personnel.

Some of these devices, such as the thermometer and otoscope are well recognized as disease vectors, and are commonly used with disposable elements or covers to prevent transmittal of microorganisms. For other devices, such as the stethoscope, protective covers are more difficult to implement. Disposable stethoscopes are expensive and are compromised in quality. Manual sterilization with disinfectant chemicals is sometimes done, but this is time consuming and not performed as often as is desirable. The hands and clothing of healthcare workers typically are sterilized by washing, but this is often inconvenient and time consuming.

U.S. Pat. No. 5,892,233, which issued to Richard T. Clement on Jan. 26, 1996, describes a portable stethoscope sterilizer which uses UV light. This device requires the stethoscope to be held by the device during a lengthy period of sterilization and, therefore, the sterilizer to be carried along with the stethoscope. Thus, a separate device is needed for each stethoscope and the healthcare worker must carry the sterilizer as they work, which is inconvenient.

As should be appreciated from the foregoing, there exists a need for improved systems and methods of sterilization or disinfection.

### SUMMARY OF THE INVENTION

One embodiment of the invention is directed to a sterilizer/disinfector for sterilizing or disinfecting an object. The sterilizer/disinfector includes a housing, a light source disposed within the housing, a light seal to block light output from the light source from exiting the housing, wherein the object forms part of the light seal, and an actuator, triggered by detection of completion of the light seal to a certain degree, to permit light to be output from the light source.

Detection of completion of the light seal to a certain degree can be accomplished in a number of different ways. For example, a device can be used which detects mechanical positions of elements that form the seal. Alternatively, an optical device can detect the degree of the light seal within the housing.

Another embodiment of the invention is directed to a method of sterilizing or disinfecting an object comprising: introducing at least a first portion of the object into a sterilizer/disinfector; sealing light within the sterilizer/disinfector using at least a second portion of the object to form a light seal; and automatically, upon detection of completion of the light seal to a certain degree, flash an ultraviolet light onto the at least a second portion of the object within the sterilizer/disinfector.

Another embodiment of the invention is directed to a device including: a housing having an opening for receiving an object; at least one movable member, attached to the housing, the at least one movable member movable between an open position and a closed position; an ultraviolet light source within the housing; and a detector that detects at least one of: (1) a degree of light sealing of the housing caused at least in part by the movable member, (2) the movable member being in the closed position, and (3) an object being located in a certain position at least partially within the housing; wherein, when the object is placed at least partially within the housing, the movable member is in the closed position, and the detector detects the at least one of (1) a degree of light sealing of the housing caused at least in part by the movable member, (2) the movable member being in the closed position, and (3) an object being located in a certain position at least partially within the housing, then the ultraviolet light source emits UV radiation to sterilize or disinfect the object.

Another embodiment of the invention is directed to a device comprising: a housing having an opening to receive at least partially an object; at least one movable member, attached to the housing, the at least one movable member movable between an opened position and a closed position; an ultraviolet light source within the housing; and an actuator that prevents the ultraviolet light source from emitting ultraviolet radiation until the object is placed at least partially within the housing and the movable member is in its closed position.

### BRIEF DESCRIPTION OF THE DRAWINGS

FIGS. 1-7 are diagrams illustrating a sterilizer/disinfector according to one embodiment of the invention;

FIGS. 8A and 9 are diagrams illustrating a light-tight seal according to one embodiment of the invention;

FIG. 8B is a diagram illustrating a cross-sectional view along line A-A of FIG. 8A;

FIGS. 10A-C are diagrams illustrating vanes of a sterilizer/disinfector according to another embodiment of the invention;

FIGS. 11-14 are diagrams illustrating a sterilizer/disinfector according to another embodiment of the invention;

FIGS. 15-17 are diagrams illustrating a sterilizer/disinfector according to a further embodiment of the invention;

FIGS. 18-20 are diagrams illustrating a sterilizer/disinfector according to another embodiment of the invention;

FIG. 21 is a block diagram of an electrical circuit for use in any of the described sterilizer/disinfector embodiments; and

FIG. 22 is a diagram illustrating an electrical circuit for use in any of the described sterilizer/disinfector embodiments.

### DETAILED DESCRIPTION

Overview of the Invention

There is a need for a technique for rapidly sterilizing peoples' hands and/or medical and hygienic devices, such as stethoscopes, particularly in the healthcare setting. The sterilization technique should be easy to use and very fast for greater user compliance. It should not use chemicals that need to be dried or removed, and it should not use heat, as some devices such as stethoscopes would be damaged by the high temperature needed for sterilization.

One embodiment of this invention is directed to a rapid, easy-to-use, sterilizer/disinfector for hands, clothing, and hand-held or other small devices that uses intense ultraviolet (UV) light to kill microorganisms (e.g., bacteria, viruses, etc.). This sterilizer/disinfector can be used in a few seconds, does not require any chemicals that need to be replenished or removed from the device, and does not damage the object to be sterilized/disinfected with high temperature. In addition, this invention can heat the device to be sterilized/disinfected slightly (less than 20 degrees F.), which is usually considered an advantage for devices that come in direct contact with patients. This device can be powered from small batteries, and thus be completely portable. The device can also be fixed-mounted to a wall or cart and/or powered from an AC line, as the entire sterilization procedure may require a sterilization time of a only few seconds (e.g., 1-3 seconds) or less than 1 second (e.g., 1 millisecond or 100 microseconds).

Sterilizer/disinfectors of this type can be made in a variety of configurations for specific purposes, or for general-purpose applications. For example, a special purpose device can be made expressly for sterilizing stethoscopes, and may be mounted to a wall or cart in a patient room or exam room. The sterilizer/disinfector may be designed with a housing to enclose the UV light source and prevent damage to the eyes of people nearby. A single sterilizer/disinfector may be designed to accommodate several different devices. While the sterilizers/disinfectors of various embodiments described herein suggest possible sterilization/disinfection applications (e.g., stethoscopes, thermometers, drinking glasses), many other applications are possible in accordance with the invention. For example, the sterilizers/disinfectors described may be used for sterilizing/disinfecting pulse oximeters, toothbrushes, otoscopes, blood pressure meters, dental picks, and other devices used by doctors, nurses, dentists, hygienists, other medical and dental personnel. Individuals may also use the sterilizers/disinfectors for a variety of medical, dental, and hygienic purposes.

The devices to be sterilized/disinfected may include on their surface UV light-sensitive material that changes color after exposure to UV light to indicate successful sterilization. Materials of this type are available that will return to their original color after a few minutes for indication of the next sterilization cycle. Further, patches of material that change color permanently after exposure to UV light may be included on the surface of the device to indicate the total lifetime exposure to UV. The color of the patch may indicate when it is time to replace the device.

Sterilizer/Disinfector Operation

The sterilizer/disinfector can operate in one of two modes, or using any combination of the two modes. One mode involves disinfecting the surface of an object by flooding it with high intensity ultraviolet light. Light with a wavelength in the range of 160 to 300 nanometers is lethal to microorganisms. A total exposure of about 10 milliwatt-seconds of ultraviolet light energy per square centimeter will typically sterilize/disinfect a surface. Greater or lesser amounts may be required depending on the exact characteristics of the surface and the environmental conditions, such as the temperature. The second mode involves raising the surface temperature of an object to be sterilized/disinfected to a temperature that is lethal to the microorganisms. Flooding the object's surface with ultraviolet light will raise the temperature of the object. The increased temperature will also increase the effectiveness of the ultraviolet light sterilization.

Some embodiments of this invention can use both modes of sterilization/disinfection simultaneously by illuminating the object to be sterilized/disinfected with a high intensity lamp, such as a xenon strobe light, that produces enough energy to heat the surface of the object ,to be sterilized/disinfected in addition to providing UV light. Xenon strobe lamps normally produce light across the spectrum of wavelengths between 160 and 2000 nanometers. For conventional applications of the xenon strobe, ultraviolet light having a wavelength of less than 380 nanometers is not desired, so a glass envelope around the xenon gas is designed to filter the light in this range. However, for sterilizer/disinfector applications, a xenon lamp with an envelope of ultraviolet-transmitting glass, or other substance such as fused quartz, may be used to maximize the output of sterilizing/disinfecting ultraviolet light. The ultraviolet light and the light emitted in the visible and infrared range (380 to 2000 nanometers) will provide a significant amount of energy for instantaneous heating of the surface of the object to be sterilized/disinfected for more effective sterilization/disinfection in a short time. A short impulse of radiant energy will cause heating of the surface of the object so rapidly as to not heat the interior of the object. This requires far less energy than heating the entire object and will have less effect on the structural integrity of the object such as would be caused by the melting of plastic. Human skin exposed to this light would experience only a slight warming feeling as the surface heat is quickly dissipated into the body.

Using this flash lamp technique, small objects such as a stethoscope head could be sterilized/disinfected with a total power to the xenon strobe lamp in the range of 20 to 200 joules. This amount of energy is similar to that of standard camera flash units. Flash lamps that are operated at a higher current density in xenon gas, as is the case in xenon short-arc lamps, produce a higher percentage of output light in the ultraviolet spectrum (a wavelength of 160 to 380 nanometers) for more efficient operation in a sterilizer/disinfector application. Sterilization/disinfection may be accomplished with continuous or pulsed UV sources. Advantageously, less power per flash is required in UV sources that provide pulsed light rather than continuous light.

Alternatively, sterilization/disinfection can be accomplished with other ultraviolet light sources that provide a continuous or flashed (i.e., pulsed) ultraviolet light with wavelengths in the range of 160 to 380 nanometers. These light sources would provide continuous radiant heating of the object, resulting in a smaller temperature gradient between the surface and interior of the object and a lower surface temperature. As a result of the lower surface temperature, the object benefits less from the heating.

One Embodiment of a Sterilizer/Disinfector that may be Used with a Stethoscope

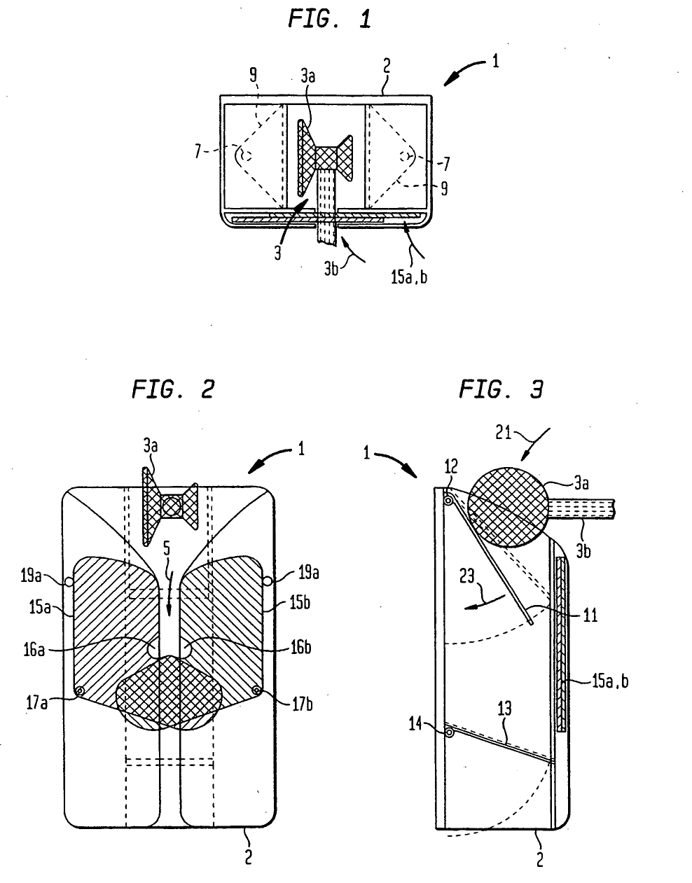

According to one aspect of the invention, a sterilizer/disinfector may be designed to sterilize and/or disinfect the head of a stethoscope, though the same sterilizer/disinfector may also be used with other devices. One illustrative embodiment of a sterilizer/disinfector that may be used to sterilize/disinfect a stethoscope is shown in FIGS. 1-7. As illustrated in FIG. 1, the sterilizer/disinfector 1 may use one or more xenon flash lamps 7 to create a flash of UV light (and/or visible and infrared light) of sufficient intensity to sterilize and/or disinfect a head 3a of a stethoscope 3 in less than 1 second (flash times of less than 1 millisecond are typical). One or more flash lamps 7 may be arranged in a housing 2, along with reflectors 9, to direct light produced by flash lamp 7, to intercept all surfaces of stethoscope 3 that are desired to be sterilized/disinfected, typically those of a head 3a at the end of a tube 3b of stethoscope 3. In the case of an electronic stethoscope, tube 3b may be a tubular structure including wires. Reflectors 9, light seal doors 11 and 13, vanes 15a and 15b, and other components that may be incorporated into sterilizer/disinfector 1, as well as a portion of the stethoscope itself, prevent the majority of the light from reaching the user, which could be uncomfortable or possibly damaging. A portion of the stethoscope itself also blocks light output from the sterilizer/disinfector, preventing a portion of light from reaching the user.

In a preferred embodiment, housing 2 is designed in such a way that head 3a of stethoscope 3 can be swiped in a smooth motion through a slot 5 in the front of housing 2. FIGS. 1-7 show an example of how housing 2 can be constructed to contain the light flash while still allowing smooth motion through it. Housing 2 has a spring-loaded upper trap door 11, which pivots about a point 12, at the top end of housing 2. When stethoscope head 3a is moved in the direction of arrow 21 (FIG. 3), upper trap door 11 is pushed downwardly and backwardly, in the direction of arrow 23. Tube 3b, attached to the stethoscope head 3a, is guided into the top of front slot 5 by the user. Housing 2 is configured with slot 5 and vanes 15a,b to assist in guiding stethoscope head 3a and tube 3b into the correct location. If the UV illumination is distributed with sufficient intensity from all directions, the rotation of stethoscope head 3a is not critical, and it is not necessary to constrain rotation of head 3a as it is moved through sterilizer/disinfector 1. This is an important feature to accommodate a variety of different configurations and sizes of stethoscopes.

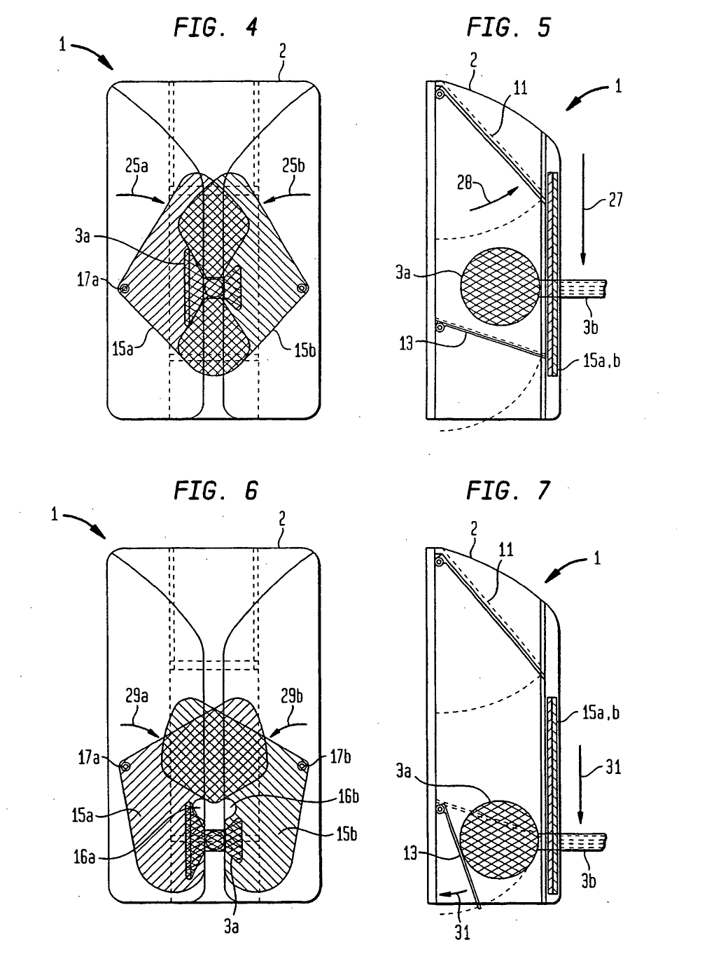

FIGS. 4 and 5 show the position of stethoscope 3 at the time of the flash for sterilization/disinfection. To reach this position, the user guides stethoscope tube 3b down within slot 5 in the front of sterilizer/disinfector housing 2, in the direction of arrow 27 (FIG. 5). Upon passing upper trap door 11 by stethoscope 3, door 11 moves in the direction of arrow 28 to return to its resting position. Vanes 15a,b are initially in the position shown in FIG. 2, against vane stops 19a,b. As tube 3b contacts vanes 15a,b in the front of housing 2, the vanes are rotated about their pivot points 17a,b. The vanes 15a and 15b are rotated in the direction of arrows 25a and 25b, respectively, and are moved against one or more return springs (not shown). At the sterilization/disinfection position, shown in FIGS. 4 and 5, the vanes have rotated so that notches 16a,b (FIG. 2) face one another, with the stethoscope tube 3b captured in the middle. A flexible seal (not shown) is built into the edges of notches 16a,b to form a light tight-seal against tube 3b when the tube is positioned in slot 5, as shown in FIG. 5. Front vanes 15a,b cover the front slot between upper trap door 11 and lower trap door 13 to form a complete light-tight housing when stethoscope 3 is in the sterilization/disinfection position.

In accordance with one embodiment, the sterilization/disinfection flash is automatically triggered when stethoscope 3 reaches a particular position in slot 5. Since the total flash time may be less than 1 millisecond (and may be as short as 100 microseconds), it is not necessary to stop the continuous movement of stethoscope 3 for sterilization/disinfection. Even with very rapid hand pulling of stethoscope 3 through slot 5, it may move less than 1/16 inch during a 1 millisecond sterilization/disinfection flash duration.

The flash triggering mechanism can be based either on the mechanical position of vanes 15a,b or on a light detector (not shown) or the like that determines when a sufficient degree of light sealing has been achieved. Some light may be emitted from the sterilizer/disinfector without exposing a user to dangerous UV levels. For example, it has been shown that a gap in a light seal having dimensions of 1/16″ by 1″ does not result in dangerous exposure levels to a user at a distance of 1′, even after hundreds or thousands of sterilization/disinfection cycles. Thus, a housing that is partially light-tight or substantially light-tight may be suitable for applications of the sterilizers/disinfectors described herein. A dark interior of housing 2 may require that the light-tight seals are in place. If there is some possibility that the sterilizer/disinfector may be used in dark environment, a light (visible or infrared, etc.) could be included on the outside of housing 2. If this light is not detected from inside housing 2, it indicates that the seals are in place. If a proper seal is not formed, flash lamp 7 is not flashed, and an error indication is made to the user so that stethoscope 3 can be passed through sterilizer/disinfector 1 again.

FIGS. 6 and 7 show the positions of sterilizer/disinfector 1 and stethoscope 3 after the sterilization/disinfection flash. The motion of stethoscope 3 may continue smoothly downwardly in the direction of arrow 31 (FIG. 7), without stopping at the sterilization/disinfection position (FIG. 4). As tube 3b is pulled though slot 5, front vanes 15a,b continue rotating about their pivot points 17a, 17b against the force of their springs. Vane 15a moves clockwise about pivot point 17a in the direction of arrow 29a; vane 15b moves counter-clockwise about pivot point 17b in the direction of arrow 29b. As vanes 15a,b rotate, stethoscope tube 3b is released from notches 16a,b in vanes 15a,b and continues moving through slot 5 in the direction of arrow 31. Head 3a of stethoscope 3 pushes lower trap door 13, against the force of its return spring, such that lower trap door 13 rotates about pivot point 14 in the direction of arrow 31. The opening of lower trap door 13 allows stethoscope head 3a to exit sterilizer/disinfector 1 though the bottom of the unit. After head 3a and tube 3b of stethoscope 3 have moved clear of sterilizer/disinfector 1, springs (not shown) cause lower trap door 13 and vanes 15a,b return to their original rest positions, as shown in FIG. 1, ready for the next sterilization/disinfection.

The embodiment of FIGS. 1-7 illustrates the sterilization/disinfection of a stethoscope head. However, it should be appreciated that the same sterilizer/disinfector could also be used with other objects that include a small neck of similar size to that of the stethoscope tube, such as a thermometer probe with the proper diameter handle, a pulse oximeter, or other medical, dental, or hygienic devices. Sterilizers/disinfectors using this same configuration may be made in different sizes to accommodate larger or smaller objects. The width of the slot and vane seals may be chosen to match the contour of desired objects, or the objects to be sterilized/disinfected can be designed to match a specific sterilizer/disinfector.

For example, a sterilizer/disinfector using this configuration could be designed to sterilize and/or disinfect a person's hand. The slot and vane seals would be designed to seal against the wrist or forearm, and would accommodate a range in sizes. The open hand would be swiped through the sterilizer/disinfector in the same fashion as was described for the stethoscope, and a UV flash would sterilize and/or disinfect the surface of the hand. For this application, it may be desirable to block the long-wave UV light (i.e., UVA and UVB in the range of 300 to 400 nm wavelength) to prevent sunburn or other skin damage resulting from repeated use. Sterilization/disinfection is accomplished primarily with UVC (i.e., wavelengths shorter than 300 nm) light. The skin is nearly opaque to UVC light. Current data appears to indicate that it is safe to use at levels that would sterilize and/or disinfect the skin surface.

Objects to be sterilized/disinfected can also be specifically modified for use in a sterilizer/disinfector of this type, for example, by including a spot of UV-sensitive material on the surface of the object. UV-sensitive materials may employ photochromic inks or pigments which may be added to a material when molded (e.g., plastic) or added as a layer on a base material. UV-sensitive material may change color in response to UV light to indicate the total exposure to UV over a short period of time and then gradually return to the original color. This type of UV-sensitive material is typically used as a dosimeter to indicate sunburn potential when exposed to sunlight. A spot of this material on the device to be sterilized/disinfected can be used as an indicator of successful exposure to UV and, therefore, successful sterilization/disinfection. When the spot has returned to its original color, it can be used as an indicator for the next sterilization/disinfection. The formulation of the UV-sensitive material or the formulation of a filter layer over it may be chosen to provide the proper color change for the desired exposure level. Even if the wavelength sensitivity of this UV sensitive material is not the same as the wavelength range UV light needed for sterilization/disinfection, this type of indicator may still be used, as the ratio of different wavelengths of light from the sterilization/disinfection light source are known, and the sensitivity can be chosen accordingly to provide the proper indication.

An indicator of lifetime UV-exposure can also be included on the device to be sterilized/disinfected. For example, a spot of material that exhibits a permanent color change when exposed to UV could be used as an indicator. This material may gradually change color over multiple exposures and may be visually compared to a reference color spot next to it. Matching colors may indicate that it is time to replace the device before significant degradation occurs. The formulation of the material or the formulation of a filter layer over it may be chosen to provide the proper color change over the total exposure desired. Even if the wavelength sensitivity of the UV-sensitive material is not the same as the wavelength range of UV light needed for sterilization/disinfection, the indicator can still work, as the ratio of different wavelengths of light from the sterilization/disinfection light source are known, and the sensitivity can be chosen accordingly to provide the proper indication.

One Embodiment of a Light-Tight Seal for a Sterilizer/Disinfector

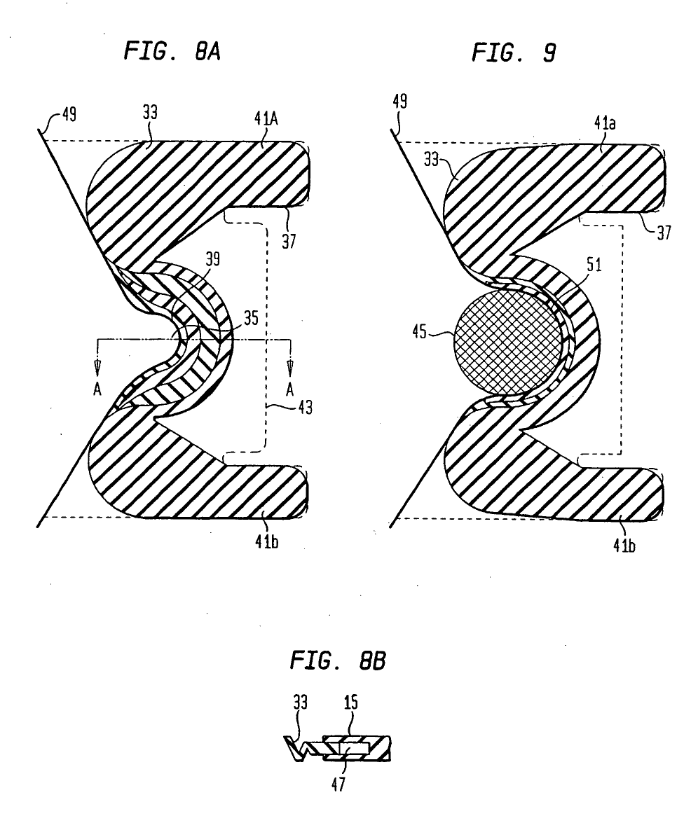

FIGS. 8A and 9 show an illustrative embodiment of a compliant, light-tight seal 33 that may be used around a central hole 35 between vanes 49 in a sterilizer/disinfector configuration. The seals on each vane 49 may be made from a compliant elastomeric material and may be installed as mirror images in a recess 37 in the edge 43 of each vane 15. FIG. 8B illustrates a cross-sectional view along line A-A of FIG. 8A, and shows an aspect of the invention in which seal 33 may fit within a pocket 47 of vane 49. Seals 33 are designed to accommodate objects having a range of sizes and shapes, and each may have a small internal radius 39 (FIG. 8A) to accommodate small stethoscope tubes or devices with a small neck. Convolution in the material near hole 35 is designed to allow the material to easily stretch around a larger diameter 51. Each seal must be in contact with over at least half of the circumference of a device 45. To maintain the seal in contact with device 45, tension is maintained in the elastomeric material on the outside of the convolution. This tension is controlled by the cantilever mounting of the top and bottom anchor points 41a,b of seal 33. The flexure of the material as larger diameters are inserted creates the tension which bends the cantilever section toward the hole. The flex points of the cantilever sections are significantly above and below the edges of the hole, so the tension causes the cantilever section to press inward against the top and bottom of the tube to keep the seal in contact with the tube in these areas.

The embodiment of FIGS. 8 and 9 is one example of a seal design, which is made from a solid elastomer and achieves its high compliance from the shape of the material. Seals made with foamed elastomer material or from low durometer (highly flexible) materials may be made with simpler geometry, but at the expense of reduced durability and longevity of use. Simpler seals may also be used in applications where a small amount of light leakage is tolerable and/or the device to be sterilized/disinfected is of a standard size, or is designed to seal easily to a specific mechanical configuration.

Alternate Embodiment of Vanes for a Sterilizer/Disinfector

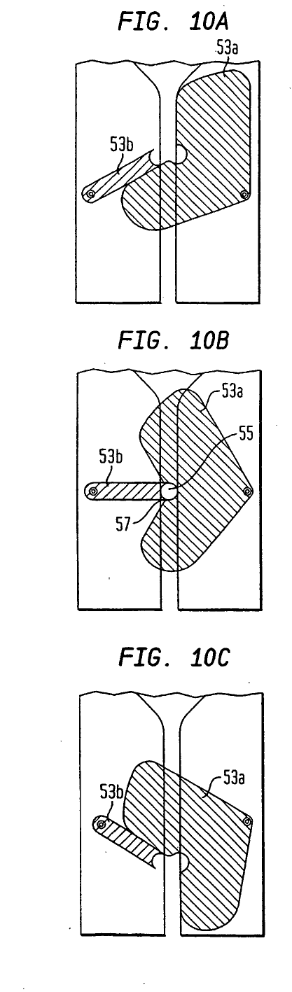

An alternative embodiment of a pass through sterilizer/disinfector for similar applications uses front vanes that are in the same plane, rather than overlapping. An example of this configuration is shown in FIGS. 10A-C. According to this embodiment, vanes 53 include a larger vane 53a and a smaller vane 53b. As shown in FIG. 10B, since vane 53a covers nearly all of the slot, except for a small area 57 next to opening 55, the smaller vane 53b only needs to be large enough to fill this small area. A compliant seal (not shown) may be included on the end of smaller vane 53b that meshes with the seal on larger vane 53a to create a complete light-tight seal. A mechanical coupling between the vanes 53 may also be included to keep the vanes moving together.

Alternate Embodiment of a Sterilizer/Disinfector that may be Used with a Stethoscope

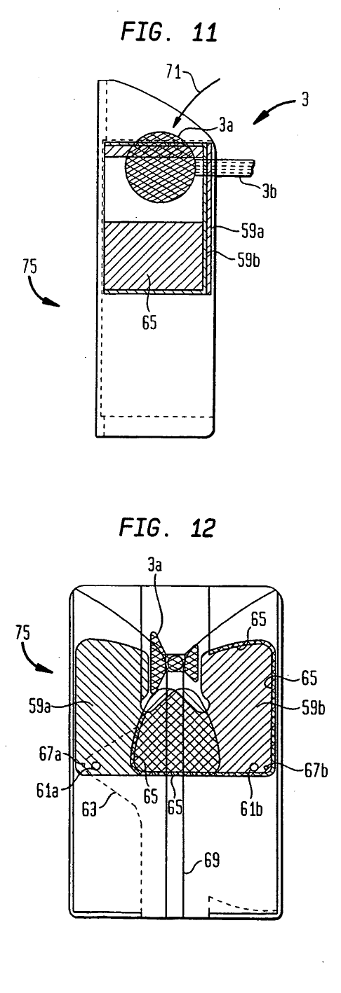

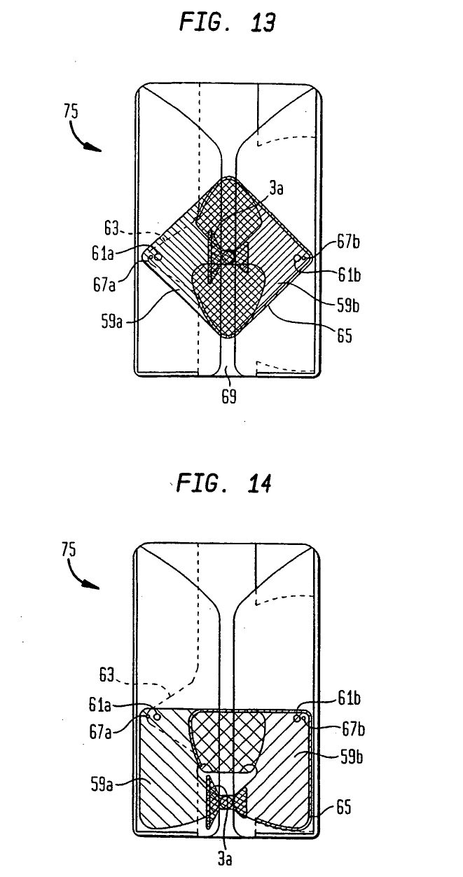

FIGS. 11-14 show an illustrative embodiment of a pass-through sterilizer/disinfector that uses extensions on one of the front vanes to replace the need for top and bottom trap doors, described above. A sterilizer/disinfector 75 according to this embodiment includes a left vane 59a and a right vane 59b, which respectively pivot about points 67a and 67b. As shown in FIG. 13, a sterilization/disinfection compartment is formed from walls 65 attached to right vane 59b. As shown in FIG. 12, an opening in these walls is initially facing upwardly when vanes 59 are held in the rest position by return springs (not shown). Stethoscope head 3a, or another object to be sterilized/disinfected, is placed into the opening, as shown by arrow 71 (FIG. 11). Tube 3b from the stethoscope 3 protrudes through a front slot 69 of sterilizer/disinfector 75.

The user pulls stethoscope 3, or another object to be sterilized/disinfected, downwardly to a sterilization/disinfection position, shown in FIG. 13. In this position, the walls 65 of right vane 59b interface with a reflector 63 around a flash lamp 61a on the left side of sterilizer/disinfector 75 to form a light-tight seal. The interior of walls 65 of right vane 59b may include a reflective coating to direct light from a flash lamp 61b on the right side of sterilizer/disinfector 75. UV light flashes at this point from flash lamps 61a,b to sterilize and/or disinfect the object.

As stethoscope 3 is pulled downwardly through slot 69, vanes 59 continue to rotate until an opening in walls 65 of right vane 59b is at the bottom of the unit, as shown in FIG. 14. Stethoscope 3 continues moving downwardly through slot 69 and out through the bottom of sterilizer/disinfector 75. Vanes 59 are spring loaded to return to their original resting positions when the stethoscope or other object is removed. This configuration requires fewer moving components than the embodiments of FIGS. 1-7, but places additional mechanical constraints on the size and shape of the sterilization/disinfection region and may not be suitable for some applications.

One Embodiment of a Sterilizer/Disinfector that may be Used with a Thermometer

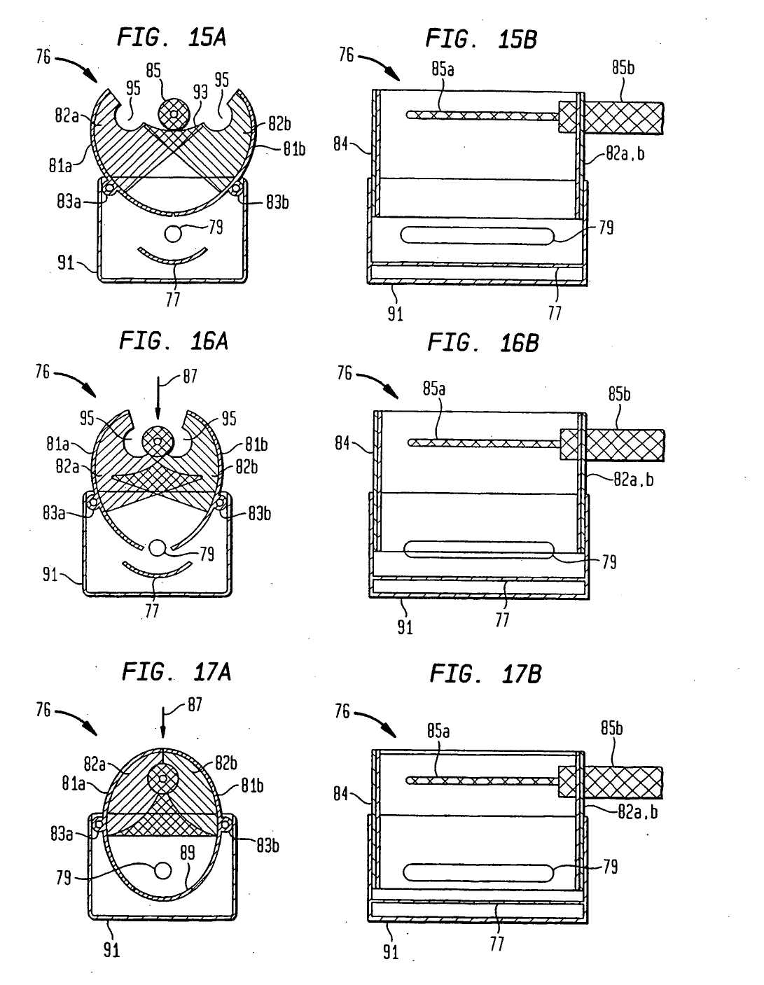

FIGS. 15-17 show an illustrative embodiment of a sterilizer/disinfector that may employ a UV flash and wherein the object to be sterilized/disinfected is pushed in and then pulled back out along the same path, and from the same side, of the sterilizer/disinfector. FIGS. 15A, 16A, and 17A sequentially show front views of the sterilizer/disinfector as the object in inserted; FIGS. 15B, 16B, and 17B show corresponding side views of FIGS. 15A, 16A, and 17A, respectively. Sterilizer/disinfector 76 includes a base 91 that is coupled to clam-shell style doors 81a and 81b via pivots 83a and 83b, respectively. Doors 81a and 81b are held open by springs (not shown), and include front door members 82a and 82b and one or more rear door members 84. Each of front door members 82a and 82b contains a notch 95a,b to accommodate an object to be sterilized/disinfected when the doors come together, as shown in FIG. 17A, and is offset from one another so as to occupy an adjacent, but separate plane from the other. Further, front door members 82a and 82b are shaped such that when an object to be sterilized/disinfected is pressed against an overlap region 93 of doors 81, the doors pivot towards one another as shown in FIG. 16A. Doors 81 form a solid wall and complete closed compartment when the doors are closed. Base 91 includes at least one flash lamp 79, and at least one reflector 77 that may be curved to direct light from flash lamp 79 upwards toward the object being sterilized/disinfected.

Front door members 82a,b of doors 81, which enclose the object to be sterilized/disinfected within notches 95, are actuated by a portion of the device to be sterilized/disinfected. Doors 81 close and open automatically as the object is inserted and withdrawn. It is important to ensure the sterilized/disinfected portion of the object does not come in contact with a non-sterile surface such as the outside surface of the sterilization/disinfection compartment during insertion or withdrawal. FIGS. 15-17 show the object to be sterilized/disinfected as a thermometer 85 having a probe 85a and handle 85b, though other objects such as a toothbrush, dental pick, or other medical, dental, or hygienic devices may be used with the sterilizer/disinfector of this embodiment. As shown, the handle 85b actuates doors 81, while probe 85a is contained within sterilizer/disinfector 76.

Continued pressing on thermometer handle 85a in the direction of arrow 87 causes it to move closer to flash lamp 79 and causes doors 81 to close by coming together at the top, as shown in FIG. 17A. When the doors are open, a safety interlock (not shown) may prevent a flash of UV light. When the doors are closed, the safety interlock may allow a flash of UV light from flash lamp 79 to sterilize and/or disinfect the probe. The safety interlock can be implemented with mechanical and/or optical sensors. After the sterilization/disinfection flash, probe 85a can be lifted away from the sterilizer/disinfector by reversing the motion of insertion. According to one embodiment, doors 81 will open automatically as probe 85a is moved back.

When the doors are completely closed, a reflective surface 89 (FIG. 17A) on the inside of doors 81 and reflector 77 below flash lamp 79 form a complete elliptical reflector, with flash lamp 79 positioned at one focus of the ellipse and probe 85a at the other focus. This shape provides optimum UV light transfer from flash lamp 79 to probe 85a and allows probe 85a to be illuminated from all sides. Doors 81 may include a compliant seal, or an interleaving seal such as a tongue-in-groove joint, along the mating edges to prevent light leakage. When doors 81 are in an open (rest) position, the doors can be designed (as shown in FIG. 15A) such that the bottom edges of doors 81 come together in front of flash lamp 79 to protect lamp 79 and reflector 77 and keep them clean.

A thermometer probe is an example of one object that may be sterilized/disinfected according to the above-described embodiment. A sterilizer/disinfector may be used with many objects other than thermometer probes in accordance with the invention. Further, many variations on sterilizer/disinfector 76 are possible, including detents to hold the doors open and/or closed, and variations in the seal designs along the edges of the doors and between the doors and the device to be sterilized/disinfected.

One Embodiment of a Sterilizer/Disinfector that may be Used with a Drinking Glass

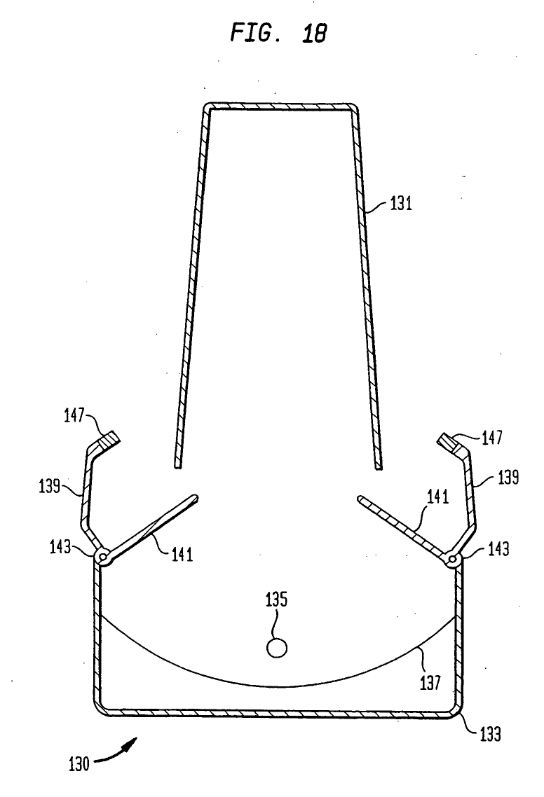

FIGS. 18-20 show an illustrative embodiment of a UV flash sterilizer/disinfector that may be used to sterilize or disinfect a container such as a drinking glass 131. Advantageously, the sterilizer/disinfector of this embodiment allows a container to be introduced into and withdrawn from the sterilizer/disinfector in a single motion. Further according to this embodiment, the action of introducing the container may actuate the sterilization or disinfection mechanism (e.g., flash of UV light), and the container itself, or other object introduced for sterilization/disinfection, may form part of a light seal that prevents light from the disinfection/sterilization flash from escaping from the confines of the sterilizer/disinfector. In the embodiment of FIGS. 18-20, sterilizer/disinfector 130 includes a base 133, a flash lamp 135 and a reflector 137 within base 133, a pair of light seals 139, and a pair of light-seal actuators 141 that are pivotally attached to base 133 via hinge mechanisms 143.

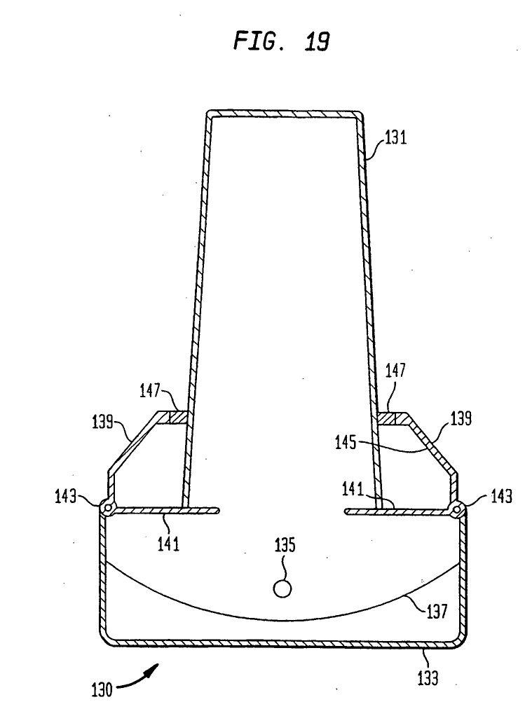

Flash lamp 135 may emit a flash of UV light, or light from another portion of the electromagnetic spectrum, for sterilization/disinfection. Light emitted downwardly by flash lamp 135 is redirected upwardly by reflector 137 towards drinking glass 131, or another object being sterilized or disinfected. Drinking glass 131 may be inserted as shown in FIG. 18 so that the rim of the glass contacts light-seal actuators 141, which are angled upwardly in their resting position. As the drinking glass in pushed against light-seal actuators 141, light seals 139 and light-seal actuators 141 rotate inwardly towards base 133 about hinge mechanisms 143. Hinge mechanisms 143 may include springs to provide resistance against the rotation motion, such that the resting position of light seals 139 and light-seal actuators 141 is as shown in FIG. 18. Each of light seals 139 may include a compliant seal portion 147, made of foam, rubber, flexible plastic, or any other suitable compliant material. Compliant seal portions 147 are disposed at the end of light seals 159, and interface with drinking glass 131 when the drinking glass is fully inserted and light-seal actuators 141 are fully depressed, as shown in FIG. 19.

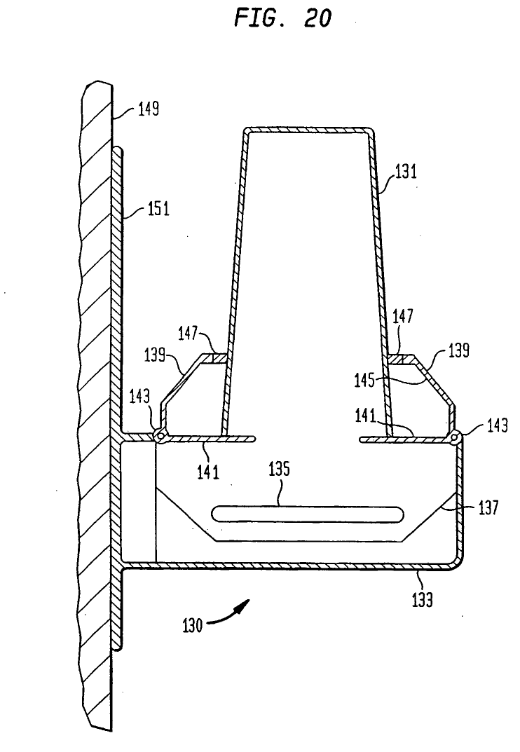

A trigger mechanism (not shown) may be included in sterilizer/disinfector 130 to initiate the light flash from flash lamp 135 when light-seal actuators 141 are fully depressed. Alternatively, a light flash from flash lamp 135 may be initiated when the glass is detected to be in the proper position, when the light seal is detected to be substantially complete, or when the user activates a switch. Light-seal actuators 141 may be transparent to UV light so that light emitted by flash lamp 135 may pass through the light-seal actuators to contact drinking glass 131. Light seals 139 may include reflective surfaces 145 to redirect light that has passed through light-seal actuators 141 downwardly and inwardly, towards the exterior rim of drinking glass 131. Drinking glass 131 may be opaque so as to prevent light emitted by flash lamp 135 from escaping from the confines of the sterilizer/disinfector, and thereby minimize potential UV light exposure to a user. The light emitted by flash lamp 135, for purposes of disinfection/sterilization, may have a duration of less than one second, allowing drinking glass 131 to be withdrawn almost immediately after introduction, if desired. Alternatively, drinking glass 131 may be retained in disinfector/sterilizer 130 for storage. When drinking glass 131 is removed, light seals 139 and light-seal actuators 141 may return automatically to their resting position, shown in FIG. 20. As shown in FIG. 20, base 133 of sterilizer/disinfector 130 may include a wall mountable portion 151 that may be affixed to a wall 149 via screws, adhesive, nails, magnets, or any other mounting means, for convenient storage of sterilizer/disinfector 130.

Sterilizer/Disinfector Electrical Configuration

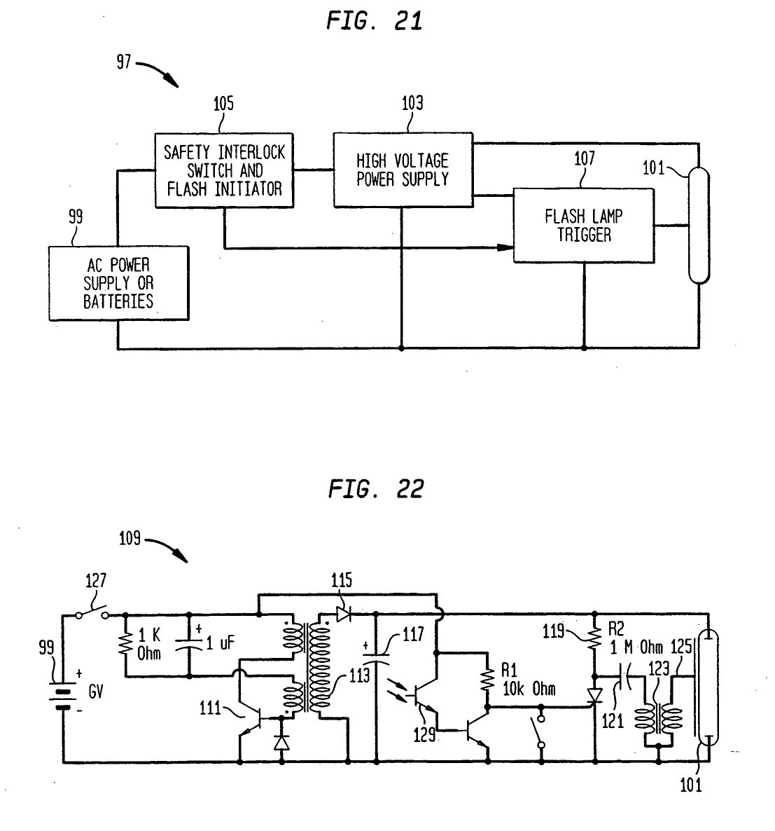

According to one embodiment of the invention, electrical circuitry associated with a flash lamp of a sterilizer/disinfector may be implemented as shown by electrical circuit 97 in FIG. 21. Electrical circuit 97 may be used in a sterilizer/disinfector according to any of the embodiments described above. Electrical circuit 97 uses a high voltage power supply 103 that contains a capacitor to store the energy necessary to power a flash lamp 101. A power source 99, which may be an AC line or a battery, typically supplies a voltage in the range of 200V to 1000V depending characteristics of the flash lamp used, though the voltage supplied may be smaller than 200V or greater than 1000V. Small linear flash lamps typically operate with voltages of 200V to 500V; small short-arc flash lamps may require 1000V or more. The voltage is selected based on the flash lamp specifications: the total energy desired per flash and the maximum flash current desired. A higher voltage will provide a higher flash current for the same energy, resulting in a greater percentage of the flash light output in the ultraviolet spectrum. The energy per flash is determined by Equation 1:
E= 1/2 CV² [1]
where E is the energy per flash in Joules, C is the value of the energy storage capacitor in Farads and V is the voltage in volts. For a sterilizer/disinfector application, the selected voltage should be as high as possible so that the flash lamp produces the greatest amount of ultraviolet light. The value of the capacitor is then chosen to provide the desired amount of energy per flash. The total energy required for this application will depend on the size of the object to be sterilized/disinfected, and will typically be in the range of 20 duration of less than one second, allowing drinking glass 131 to be withdrawn almost immediately after introduction, if desired. Alternatively, drinking glass 131 may be retained in disinfector/sterilizer 130 for storage. When drinking glass 131 is removed, light seals 139 and light-seal actuators 141 may return automatically to their resting position, shown in FIG. 20. As shown in FIG. 20, base 133 of sterilizer/disinfector 130 may include a wall mountable portion 151 that may be affixed to a wall 149 via screws, adhesive, nails, magnets, or any other mounting means, for convenient storage of sterilizer/disinfector 130.
Sterilizer/Disinfector Electrical Configuration

According to one embodiment of the invention, electrical circuitry associated with a flash lamp of a sterilizer/disinfector may be implemented as shown by electrical circuit 97 in FIG. 21. Electrical circuit 97 may be used in a sterilizer/disinfector according to any of the embodiments described above. Electrical circuit 97 uses a high voltage power supply 103 that contains a capacitor to store the energy necessary to power a flash lamp 101. A power source 99, which may be an AC line or a battery, typically supplies a voltage in the range of 200V to 1000V depending characteristics of the flash lamp used, though the voltage supplied may be smaller than 200V or greater than 1000V. Small linear flash lamps typically operate with voltages of 200V to 500V; small short-arc flash lamps may require 1000V or more. The voltage is selected based on the flash lamp specifications: the total energy desired per flash and the maximum flash current desired. A higher voltage will provide a higher flash current for the same energy, resulting in a greater percentage of the flash light output in the ultraviolet spectrum. The energy per flash is determined by Equation 1:
E= 1/2 CV² [1]
where E is the energy per flash in Joules, C is the value of the energy storage capacitor in Farads and V is the voltage in volts. For a sterilizer/disinfector application, the selected voltage should be as high as possible so that the flash lamp produces the greatest amount of ultraviolet light. The value of the capacitor is then chosen to provide the desired amount of energy per flash. The total energy required for this application will depend on the size of the object to be sterilized/disinfected, and will typically be in the range of 20 to 200 joules for small objects such as a stethoscope head. The energy requirement is a function of how efficiently the light from the flash lamp is directed to the object to be sterilized/disinfected, the size and surface characteristics of the object, and the spectrum of light from flash lamp 101.

The sterilizer/disinfector circuitry also includes a flash lamp trigger 107 which is very similar to the trigger circuit in a camera flash. The flash lamp trigger provides a very high voltage pulse, typically in the range of 4 kV to 15 kV depending on the specifications of the flash lamp, to initiate the flash. According to one embodiment of the sterilizer/disinfector, a charge storage capacitor is kept charged to the appropriate voltage whenever the unit is powered on. Flash lamp trigger 107 is initiated when the object to be sterilized/disinfected is in the correct position and a safety interlock 105 indicates that the sterilization/disinfection chamber is closed and light-tight. Safety interlock 105 prevents triggering of flash lamp 101 when the sterilizing compartment is open, and indicates an error condition to the operator.

FIG. 22 shows one example of a typical battery powered xenon flash lamp driver circuit with trigger circuitry for activating a flash lamp. Circuits of this nature are commonly used in camera flash units. For simplicity, the diagram does not show the details of an AC power supply or user indicators. A power transistor 111 and its related components form a low voltage oscillator, typically in the range of 15 to 20 kHz. Current from a high voltage transformer 113 passes through a high voltage diode 115 and charges an energy storage capacitor 117 to a voltage that will drive flash lamp 101. A resistor 119 charges a trigger capacitor 121 to the flash lamp voltage. When the SCR is turned-on, trigger capacitor 121 is discharged through a trigger transformer 123 which creates a very high voltage pulse to a trigger electrode 125 on flash lamp 101, causing it to flash using the stored energy in energy storage capacitor 117. The SCR is turned-on only when a safety interlock switch 127 (mechanically connected to the front vanes) is open, signifying that the vanes are in the proper position for the sterilization/disinfection, and when there is no light falling on a phototransistor 129 that is placed inside the sterilizing compartment.

No separate user controls for the sterilizer/disinfector are needed except for an on-off switch to control the power to the unit. The energy storage capacitor is charged automatically to the desired voltage (in the same way a camera flash charges), and maintained there until the sterilizer/disinfector is activated by passing an object through it. The control circuit could include one or more indicators, such as light emitting diodes and/or audio beepers to indicate that the device is ready, or to indicate that it failed to flash because of a light leak to the sterilization/disinfection compartment. An indicator could tell the user when the sterilization/disinfection is completed successfully.

It should be appreciated that the above-described circuitry is merely intended to illustrate one possible implementation, and many such circuits are possible and known in the art. For example, there exists in the art many circuits for driving flash lamps that may be suitably applied to the sterilizers/disinfectors described herein. Thus, the invention is not limited in this respect.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and equivalents thereto.

1. A method of sterilizing or disinfecting an object, comprising acts of:
introducing at least a first portion of the object into a sterilizer/disinfector;
sealing light within the sterilizer/disinfector using at least a second portion of the object to form a light seal; and
automatically, upon detection of completion of the light seal to a certain degree, flashing ultraviolet light onto the at least a first portion of the object within the sterilizer/disinfector.

2. The method of claim 1, further including an act of killing microorganisms on the object.

3. The method of claim 2, wherein the act of automatically flashing includes automatically flashing ultraviolet light for a duration of less than one second.

4. The method of claim 2, wherein the act of automatically flashing includes automatically flashing ultraviolet light for a duration of less than 10 milliseconds.

5. The method of claim 2, wherein the act of automatically flashing includes automatically flashing pulsed ultraviolet light.

6. A method for sterilizing or disinfecting an object, comprising acts of:
continuously moving at least a portion of the object through a chamber including an ultraviolet light source;
actuating movement of a light seal, using the object; and
applying ultraviolet light to the at least a portion of the object, with an intensity sufficient to sterilize or disinfect the at least a portion of the object, as it moves past the ultraviolet light source.

7. The method of claim 6, wherein the act of applying includes applying ultraviolet light to the object automatically in response to a signal triggered by a position of the object.

8. The method of claim 6, wherein the act of continuously moving includes continuously moving at least a portion of a stethoscope through a chamber.

9. The method of claim 6, wherein the act of continuously moving includes continuously moving at least a portion of a pulse oximeter through a chamber.

10. The method of claim 6, wherein the act of continuously moving includes continuously moving at least a portion of a device through a chamber, the device being selected from the group consisting of a medical device, a dental device, and a hygienic device.

11. A method of sterilizing or disinfecting at least a portion of the object, comprising acts of:
introducing the at least a portion of the object into a housing;
actuating rotation of two or more vanes pivotally mounted to the housing, using the object;
orienting the two or more vanes to form an opening when the object is at a sterilization/disinfection position in which a portion of the object is located;
applying ultraviolet light to the object at the sterilization/disinfection position.

12. The method of claim 11, wherein the act of introducing includes introducing at least a portion of a stethoscope into a housing.

13. The method of claim 12, wherein the act of orienting includes orienting the two or more vanes to form a substantially circular opening.

14. The method of claim 11, wherein the act of orienting includes orienting the two or more vanes to form an opening that is shaped to accommodate the object.
